(19) United States
(12) Patent Application Publication
(10) Pub. No.: US 2026/0258494 A1
(43) Pub. Date: Sep. 3, 2026

(54) SPATIAL OMICS CHIP AND MANUFACTURING METHOD THEREFOR, AND SYNTHESIS METHOD FOR SPATIAL POSITION TAG SEQUENCE AND SAMPLE INTAKE CHIP THEREOF

(71) Applicant: BOE Technology Group Co., Ltd., Beijing (CN)

(72) Inventor: Jian YAN, Beijing (CN)

(21) Appl. No.: 18/881,742

(22) PCT Filed: Mar. 20, 2024
(86) PCT No.: PCT/CN2024/082614

§ 371 Date: Jan. 7, 2025

(30) Foreign Application Priority Data
Apr. 28, 2023 (CN) 202310485391.X

(51) Int. Cl. C12Q 1/6874; C12Q 1/6876

(52) U.S. Cl. CPC C12Q 1/6874; C12Q 1/6876

(57) ABSTRACT

A spatial omics chip and a manufacturing method therefor, and a synthesis method for a spatial position tag sequence and a sample intake chip thereof. The spatial omics chip comprises: a first substrate (10), hydrophobic regions (20), which are arranged on the same side of the first substrate (10), and a plurality of hydrophilic regions (30), which are distributed in a periodic array and are spaced apart from each other, wherein each hydrophilic region (30) is surrounded by the hydrophobic regions (20), and each hydrophilic region (30) comprises a hydrophilic structure (31), the hydrophilic structure (31) being configured to be connected to a coding probe (40).

### CROSS-REFERENCE TO RELATED APPLICATIONS

The present application is a U.S. National Phase Entry of International Application No. PCT/CN2024/082614 having an international filing date of Mar. 20, 2024, which claims the priority to Chinese patent application No. 202310485391.X, filed to CNIPA on Apr. 28, 2023, and entitled “Spatial Omics Chip and Manufacturing Method Therefor, and Synthesis Method for Spatial Position Tag Sequence and Sample Intake Chip Thereof”. The contents of the above-identified applications should be construed as being incorporated into the present disclosure by reference.

### TECHNICAL FIELD

Embodiments of the present disclosure relates to, but are not limited to, spatial omics technology, and particularly relate to a spatial omics chip and a manufacturing method therefor, a synthesis method for a spatial position tag sequence and a sample injection chip thereof.

### BACKGROUND

Traditional transcriptomics generally studies overall RNA expression information of a target biological tissue. The results of the studies reflect overall transcriptome expression of the biological tissue, while gene expression specificities of different cells in the tissue are ignored. The emerging single-cell transcriptomics technology can better distinguish RNA expression differences between different cells, and overcome the problem that cell heterogeneity in the same tissue cannot be discriminatively detected. The information of spatial positions between cells is closely related to the physiological and pathological conditions of biological tissues or organs. However, neither traditional transcriptomics nor single-cell transcriptomics technologies can reveal the spatial heterogeneity information of multicellular tissues. Therefore, the studies and application of such technologies in tissue development, organ development, and the occurrence and development mechanism of diseases such as tumors have been limited to a certain extent.

In recent years, the emerging spatial transcriptomics (ST) technology has met the two major needs of differentiated detection of cell gene expression heterogeneity and spatial position information marking in the field of biotechnology applications. Since ST technology is first published in the journal SCIENCE in 2016, it has developed into a variety of different technical implementations. These technical implementations can be basically divided into two types: ST technology based on fluorescent probes and imaging, and ST technology based on in-situ capture and sequencing. ST technology based on fluorescent probes and imaging can achieve RNA in situ detection in the true sense, but it still needs the assistance of microscopic imaging technology with higher technical barriers and complex image analysis algorithms, and its gene detection throughput is limited, tissue transparency, probe and enzyme penetration methods need to be further explored, so it is still far from commercial application. The ST technology based on in-situ capture and sequencing mainly uses specific tag sequences to perform in situ capture and spatial position marking on transcripts, and then obtain transcriptome expression information through library constructing and sequencing. It has high sensitivity and resolution, simple operation and short test cycle. At present, ST technology platforms based on in-situ capture and sequencing mainly include 10× Genomics-Visium, Slide-seq, Seq-scope, Stereo-seq, and etc. Among them, the Visium technology platform of 10× Genomics has been successfully commercialized and used most widely. The Stereo-seq technology platform, jointly developed by BGI Reserarch in Shenzhen, China and a number of institutions, also has strong technological competitive advantages due to its characteristics such as ultra-high resolution and large sample coverage area.

### SUMMARY

The following is a summary of subject matters described herein in detail. The summary is not intended to limit the protection scope of the present disclosure.

An embodiment of the present disclosure provides a spatial omics chip, containing: a first substrate, hydrophobic regions arranged on the same side of the first substrate, and a plurality of hydrophilic regions distributed in a periodic array and spaced apart from each other, wherein each hydrophilic region is surrounded by the hydrophobic regions, and the hydrophilic region contains a hydrophilic structure which is configured to be connected to a coding probe.

In an exemplary embodiment of the present disclosure, each of the hydrophilic regions may contain a plurality of hydrophilic structures distributed in a periodic array, wherein different hydrophilic structures all have a diameter of d₁, and a distance between two adjacent hydrophilic structures in each of the hydrophilic regions is 0.25d₁ to 1.5d₁.

In an exemplary embodiment of the present disclosure, a diameter d₁ of the hydrophilic structure may be 2 μm to 2.5 μm, and a height of the hydrophilic structure may be 5 μm to 10 μm.

In an exemplary embodiment of the present disclosure, the hydrophobic region may contain a plurality of hydrophobic structures, and at least one hydrophobic structure may be arranged between two adjacent hydrophilic regions; wherein different hydrophobic structures all have a diameter of d₂, and a distance between two adjacent hydrophobic structures may be 0.25d₂ to 1.5d₂, d₂ may be 2 μm to 2.5 μm; and
a distance between adjacent hydrophobic structures and the hydrophilic structures is d₃.

In an exemplary embodiment of the present disclosure, d₁=d₂, and d₃ may be 0.25d₁ to 1.5d₁.

In an exemplary embodiment of the present disclosure, a plurality of hydrophilic regions may be distributed on the first substrate in a periodic array, and a distance between centers of two adjacent hydrophilic regions is L₁, and L₁ may be not less than 1.5d₁.

In an exemplary embodiment of the present disclosure, L₁ may be 4.446d₁ to 9d₁. In an exemplary embodiment of the present disclosure, the hydrophilic structure may be connected to the first substrate by a carbon-hydrogen bond.

In an exemplary embodiment of the present disclosure, a surface of the hydrophilic structure contains an active group capable of being connected to the coding probe, and the active group can include any one or more of an amino group, a thiol group, a carboxyl group, an ester group, and an anhydride; wherein each of the hydrophobic structures contains thereon a hydrophobic group containing any one or more of a long-chain hydrocarbon group having a carbon chain length of ≥7 and a fluoroalkyl group.

In an exemplary embodiment of the present disclosure, the spatial omics chip may further contain the coding probe connected to the hydrophilic structure; wherein the coding probe may include any one or more of a chemical probe, a polypeptide probe, a fluorescent probe, and a nucleic acid probe.

In an exemplary embodiment of the present disclosure, the coding probe may be configured to capture an mRNA sample and mark a spatial position of the mRNA sample captured.

In an exemplary embodiment of the present disclosure, the coding probe contains a sequencing primer binding sequence, a spatial position tag sequence, a UMI sequence and a Poly(dT) sequence that are sequentially connected;
wherein,
the sequencing primer binding sequence is configured to be complementarily paired with a primer upon sequencing;
the spatial position tag sequence is configured to mark a spatial position of the mRNA sample captured, and the spatial position tag sequences of the coding probes connected to the hydrophilic structures located within a same hydrophilic region are same, and the spatial position tag sequences of the coding probes connected to the hydrophilic structures located in different hydrophilic regions are different; and the spatial position tag sequence contains a spatial position sub-tag sequence;
the UMI sequence is configured to specifically mark different mRNA samples; and
the Poly(dT) sequence is configured to capture the mRNA sample.

In an exemplary embodiment of the present disclosure, the sequencing primer binding sequence contains a linking group through which the sequencing primer binding sequence is connected to the hydrophilic structure, and the linking group may include at least one of a carboxyl group, a thiol group, and an amino group.

In an exemplary embodiment of the present disclosure, the sequencing primer binding sequence and the hydrophilic structure may be connected together by at least one of an amino-carboxyl reaction, a thiol-thiol reaction, and an anhydride-amino reaction.

In an exemplary embodiment of the present disclosure, the sequencing primer binding sequence and the hydrophilic structure may be connected together by at least one of an amide group and a disulfide bond.

In an exemplary embodiment of the present disclosure, the spatial omics chip may contain a plurality of sub-chips distributed at intervals, wherein the plurality of sub-chips are distributed on the first substrate in a periodic array, and the sub-chip contains a plurality of the hydrophilic regions.

An embodiment of the present disclosure also provides a rotary step-by-step sample injection chip, configured to perform by-multiple-steps sample injection into the hydrophilic regions of the spatial omics chip provided in an embodiment of the present disclosure, the rotary step-by-step sample injection chip containing: a second substrate, and a plurality of flow channels arranged on the same side of the second substrate and distributed at intervals, wherein the flow channels have a plurality of through holes penetrating the second substrate and distributed in a periodic array, each of the hydrophilic regions of the spatial omics chip corresponds to one of the through holes, a distance between the centers of two adjacent through holes is same as a distance between the centers of two adjacent hydrophilic regions corresponding to the two through holes, and the through holes is configured to perform step-by-step sample injection into each of the hydrophilic structures of the hydrophilic regions corresponding to the through holes.

In an exemplary embodiment of the present disclosure, an area of the through hole is less than or equal to an area of the hydrophilic region corresponding to the through holes.

In an exemplary embodiment of the present disclosure, the rotary step-by-step sample injection chip can contain an effective sample injection region, wherein the rotary step-by-step sample injection chip is rotated around a center thereof at least once at a set angle, causing that at least a portion of the through holes of the rotary step-by-step sample injection chip before and after at least one rotation completely overlap, and the completely overlapping through holes form the effective sample injection region, when the spatial omics chip and the rotary step-by-step sample injection chip are matched and used, an orthographic projection of the through holes in the effective sample injection region on the first substrate overlaps with an orthographic projection of the hydrophilic region of the spatial omics chip on the first substrate.

In an exemplary embodiment of the present disclosure, the effective sample injection region may be a regular polygon having n sides, and n is an integer ≥3;
a distance between centerlines of two adjacent flow channels satisfies:

$${L}_{3} = {L}_{2} \times sin θ ;$$

wherein L₃ is a distance between two adjacent flow channels;
L₂ is a distance between the centers of two adjacent through holes;
θ is a rotation angle of the rotary step-by-step sample injection chip.

An embodiment of the present disclosure also provides a synthesis method for a spatial position tag sequence, including:
injecting a sub-spatial position tag sequence multiple times through a through hole of the rotary step-by-step sample injection chip as provided in an embodiment of the present disclosure above, and between two adjacent injections, rotating the rotary step-by-step sample injection chip at a set angle around its center, such that at least part of the through holes of the rotary step-by-step sample injection chip completely overlap before and after the rotation, injecting the sub-spatial position tag sequence into a hydrophilic region of the spatial omics chip through the through holes in the overlapping region to form the spatial position tag sequence;
wherein the sub-spatial position tag sequences injected into the plurality of through holes on the same flow channel are same, and the sub-spatial position tag sequences injected into the through holes on different flow channels are different.

In an exemplary embodiment of the present disclosure, injecting the sub-spatial position tag sequence into a hydrophilic region of the spatial omics chip through the through holes of the overlapping region may include:
bonding the rotary step-by-step sample injection chip to the spatial omics chip as provided in an embodiment of the present disclosure, such that a hydrophilic region of the spatial omics chip corresponds to a through hole of a flow channel of the rotary step-by-step sample injection chip, wherein the hydrophilic structure of the hydrophilic region is connected to a nucleic acid sequence; and
injecting different sub-spatial position tag sequences into different flow channels of the rotary step-by-step sample injection chip respectively, such that the sub-spatial position tag sequences enter the hydrophilic region of the spatial omics chip through the through hole on the flow channel, and are connected to the nucleic acid sequences on the hydrophilic structures.

An embodiment of the present disclosure also provides a manufacturing method of a spatial omics chip as provided in an embodiment of the present disclosure above, including:
covering the first substrate with a first photomask having a first light-transmitting region and a first light-shielding region, wherein the first light-transmitting region completely coincides with an orthographic projection of a region on the first substrate where the hydrophilic structure is to be formed on the first substrate;
under illumination conditions, using a hydrophilic monomer as a raw material, performing a graft polymerization reaction on a region on the first substrate which completely coincides with the orthographic projection of the first light-transmitting region to form a hydrophilic polymer, wherein the hydrophilic polymer contains an active group capable of being connected to the coding probe, and forms the hydrophilic structure;
covering the first substrate with a second photomask having a second light-transmitting region and a second light-shielding region, wherein the second light-transmitting region completely coincides with an orthographic projection of a region on the first substrate where the hydrophobic structure is to be formed on the first substrate;
under illumination conditions, using a hydrophobic monomer as a raw material, performing a graft polymerization reaction on a region on the first substrate which completely coincides with the orthographic projection of the second light-transmitting region to form a hydrophobic polymer, wherein the hydrophobic polymer contains the hydrophobic group and forms a hydrophobic structure, and the hydrophobic structure forms the hydrophobic region.

In an exemplary embodiment of the present disclosure, the manufacturing method of the spatial omics chip may further include sequentially ligating a sequencing primer binding sequence, a spatial position tag sequence, a UMI sequence and a Poly(dT) sequence at an end of the hydrophilic structure away from the first substrate.

In an exemplary embodiment of the present disclosure, the spatial position tag sequence may be formed by a synthesis method of the spatial position tag sequence as provided in an embodiment of the present disclosure above.

Other features and advantages of the present disclosure will be set forth in the following specification, and moreover, partially become more apparent from the specification, or are understood by implementing the present disclosure. Other advantages of the present disclosure may be achieved and obtained through solutions described in the specification and drawings.

### BRIEF DESCRIPTION OF DRAWINGS

Accompanying drawings are used to provide understanding of technical solutions of the present disclosure, and form a part of the specification. The accompanying drawings and embodiments of the present disclosure are adopted to explain the technical solutions of the present disclosure, and do not form limitations on the technical solutions of the present disclosure.

FIG. 1A is a top view of a spatial omics chip provided in an exemplary embodiment of the present disclosure;

FIG. 1B is a section view of the spatial omics chip shown in FIG. 1A;

FIG. 2 is a top view of another spatial omics chip provided in an exemplary embodiment of the present disclosure;

FIG. 3 is a top view of yet another spatial omics chip provided in an exemplary embodiment of the present disclosure;

FIG. 4 is a top view of yet another spatial omics chip provided in an exemplary embodiment of the present disclosure;

FIG. 5 is a schematic diagram of a structure of a hydrophilic region of a spatial omics chip provided in an exemplary embodiment of the present disclosure;

FIG. 6 is a schematic diagram of a structure of a hydrophilic region of another spatial omics chip provided in an exemplary embodiment of the present disclosure;

FIG. 7 is a schematic diagram of a structure of a hydrophilic region of yet another spatial omics chip provided in an exemplary embodiment of the present disclosure;

FIG. 8 is a top view of yet another spatial omics chip provided in an exemplary embodiment of the present disclosure;

FIG. 9 is a top view of yet another spatial omics chip provided in an exemplary embodiment of the present disclosure;

FIG. 10 is a top view of a rotary step-by-step sample injection chip provided in an exemplary embodiment of the present disclosure;

FIG. 11 is a top view of another rotary step-by-step sample injection chip provided in an exemplary embodiment of the present disclosure;

FIG. 12 is a process flow diagram of a method of forming a coding sequence provided in an exemplary embodiment of the present disclosure;

FIG. 13 is a process flow diagram of another method of forming a coding sequence provided in an exemplary embodiment of the present disclosure;

FIG. 14 is a top view of a first photomask provided in an exemplary embodiment of the present disclosure;

FIG. 15 is a top view of a second photomask provided in an exemplary embodiment of the present disclosure;

FIG. 16 is a reaction mechanism diagram of graft polymerization reaction on a surface of the first substrate.

Meanings of reference signs in the accompanying drawings are as follows:

10—first substrate; 20—hydrophobic region; 21—hydrophobic structure; 30—hydrophilic region; 31—hydrophilic structure; 40—coding probe; 50—sub-chip; 51—sequencing primer binding sequence; 52—spatial position tag sequence; 521—first sub-spatial position tag sequence; 522—second sub-spatial position tag sequence; 523—third sub-spatial position tag sequence; 53—UMI sequence; 54—Poly(dT) sequence; 70—second substrate; 80—flow channel; 90—through hole; 100—effective sample injection region; 110—first background layer; 111—first light-transmitting region; 1111—first light-transmitting hole; 112—first light-shielding region; 120—second background layer; 121—second light-transmitting region; 1211—second light-transmitting hole; 122—second light-shielding region; 130—Photomask.

### DETAILED DESCRIPTION

To make objectives, technical solutions, and advantages of the present disclosure clearer, the embodiments of the present disclosure will be described in detail below with reference to the accompanying drawings. It is to be noted that the embodiments and features in the embodiments of the present disclosure may be randomly combined with each other if there is no conflict.

Implementations herein may be implemented in a plurality of different forms. Those of ordinary skills in the art can readily appreciate a fact that the implementations and contents may be varied into various forms without departing from the spirit and scope of the present disclosure. Therefore, the present disclosure should not be explained as being limited to the contents recorded in the following implementations only. The embodiments and features in the embodiments of the present disclosure may be randomly combined with each other if there is no conflict.

Scales of the drawings in the present disclosure may be used as a reference in actual processes, but are not limited thereto. For example, a width-length ratio of a channel, a thickness and spacing of each film layer, and a width and spacing of each signal line may be adjusted according to actual needs. A quantity of pixels in a display substrate and a quantity of sub-pixels in each pixel are not limited to numbers shown in the drawings. The drawings described in the present disclosure are schematic structural diagrams only, and one mode of the present disclosure is not limited to shapes, numerical values, or the like shown in the drawings.

In the specification, for convenience, wordings indicating orientation or positional relationships, such as “middle”, “upper”, “lower”, “front”, “back”, “vertical”, “horizontal”, “top”, “bottom”, “inside”, “outside”, and the like, are used for illustrating positional relationships between constituent elements with reference to the drawings, and are merely for facilitating the description of the specification and simplifying the description, rather than indicating or implying that a referred apparatus or element must have a particular orientation and be constructed and operated in the particular orientation. Therefore, they cannot be understood as limitations on the present disclosure. The positional relationships between the constituent elements may be changed as appropriate based on a direction according to which each constituent element is described. Therefore, appropriate replacements based on situations are allowed, which is not limited to the expressions in the specification.

In the specification, unless otherwise specified and defined explicitly, terms “set” and “connect” should be understood in a broad sense. For example, it may be a fixed connection, or a detachable connection, or an integral connection; it may be a mechanical connection or an electrical connection; it may be a direct connection, or an indirect connection through a middleware, or an internal communication between two elements. Those of ordinary skills in the art may understand specific meanings of the above terms in the present disclosure according to specific situations.

In description of the present disclosure, “parallel” refers to approximately parallel or almost parallel, for example, refers to a state in which the angle formed by two straight lines is above −10 degrees and below 10 degrees, and therefore also includes a state in which the angle is above −5 degrees and below 5 degrees. In addition, “perpendicular” refers to “approximately perpendicular”, for example, refers to a state in which the angle formed by two straight lines is above 80 degrees and below 100 degrees, and therefore also includes a state in which the angle is above 85 degrees and below 95 degrees.

In the description of the present disclosure, ordinal numerals such as “first” and “second” are set to avoid confusion of constituents, but not intended for restriction in quantity.

In the specification, a “film” and a “layer” are interchangeable. For example, the “hydrophilic layer” may be replaced by the “hydrophilic film” in some cases.

At present, ST technology platforms based on in-situ capture and sequencing, such as the 10× Genomics-Visium technology platform, still have low capture efficiency of sample mRNA, cross-contamination caused by lateral diffusion between adjacent regions and complicated chip processing and preparation methods, harsh conditions and other problems, and most technology platforms need to decode and sequence the spatial position tag sequence in situ previously before starting the processing of sample tissue and subsequent capture sequencing, which further increases the cost and process complexity of spatial transcriptome analysis. The current solution is mainly to prepare chip substrates (such as glass slides, etc.) with micron-sized partitioned micro-pore or micro-well reaction chamber arrays on the surface, and then put coding microspheres or microcarriers connected with unique nucleic acid molecular identifiers into them, and capture mRNA samples after tissue permeabilization, and finally complete library construction and sequencing. During tissue permeabilization, the sample surface can be covered with a porous membrane to reduce lateral contamination. However, compared with the current technology platform, the above two solutions are still not essentially different. They are both methods that rely on capturing and sequencing microspheres or microcarriers after falling into pores from the surface of porous substrates and assembling. These two solutions are faced with the problems such as limited pore falling rate of microspheres and complex flow process, and it also has no obvious effects on improving the low capture efficiency of mRNA samples, avoiding cross-contamination and simplifying chip processing methods.

Therefore, it is of great significance to develop a spatial omics chip with high capture efficiency of mRNA samples, low cross-contamination between adjacent sample regions, easy preparation, and convenient and fast operation.

An embodiment of the present disclosure provides a spatial omics chip, containing: a first substrate, hydrophobic regions arranged on the same side of the first substrate, and a plurality of hydrophilic regions distributed in a periodic array and spaced apart from each other, wherein each hydrophilic region is surrounded by the hydrophobic regions, and the hydrophilic region contains a hydrophilic structure which is configured to be connected to a coding probe.

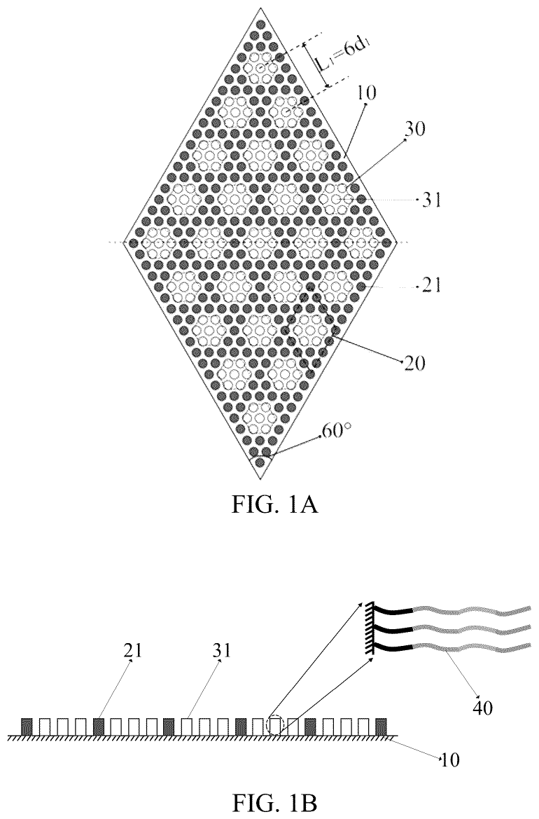

FIG. 1A is a top view of a spatial omics chip provided in an exemplary embodiment of the present disclosure; and FIG. 1B is a section view of the spatial omics chip shown in FIG. 1A.

As shown in FIG. 1A and FIG. 1B, the spatial omics chip contains a first substrate 10, hydrophobic regions 20 arranged on the same side of the first substrate 10, and a plurality of hydrophilic regions 30 distributed in a periodic array and spaced apart from each other.

A main function of the first substrate 10 is to provide a substrate support for the growing arrangement of the hydrophobic region 20 and the hydrophilic region 30, and subsequently serve as a substrate to carry the product after tissue permeabilization in order to complete a capture reaction.

A main function of the hydrophobic region 20 is to isolate all the regions 30 arranged in a periodic array and prevent cross-contamination between adjacent hydrophilic regions 30 caused by a lateral flow of samples; and the hydrophobic region 20 can be formed of a hydrophobic material.

Each hydrophilic region 30 is surrounded by the hydrophobic region 20, the hydrophilic region 30 contains a hydrophilic structure 31; and the hydrophilic structure 31 is configured to be connected to a coding probe 40. The hydrophilic region 30 can be formed of a hydrophilic material, and one end of the hydrophilic structure 31 can be connected to the first substrate 10.

In the description of the present disclosure, “surrounding” can be understood as partially surrounding or fully surrounding. In some embodiments, the surrounding can be a full surrounding, i.e. an entire periphery of the hydrophilic region is surrounded by the hydrophobic region, and the hydrophobic region surrounding the hydrophilic region is a closed annular shape. In some embodiments, the surrounding could be a partial surrounding, i.e., a portion of a periphery of the hydrophilic region is surrounded by the hydrophobic region, the hydrophobic region surrounding the hydrophilic region is not closed, for example, an annular shape with openings or a plurality of independent hydrophobic regions can form a surrounding of the hydrophilic region, as shown in FIG. 1A. In an exemplary embodiment of the present disclosure, the coding probe can include any one or more of a chemical probe, a polypeptide probe, a fluorescent probe and a nucleic acid probe (e.g., a genomic probe, a cDNA probe, a cRNA probe and an artificially synthesized oligonucleotide probe, etc.).

Exemplarily, the coding probe 40 can be configured to capture an mRNA sample and mark a spatial position of the mRNA sample captured. A main function of the hydrophilic structure 31 is to connect the coding probe to achieve capture of mRNA. The hydrophilic structure 31 has a side surface and an end face away from the first substrate 10, and both the end face and the side surface can be connected to the coding probe 40.

In an exemplary embodiment of the present disclosure, the spatial omics chip can be a proteome chip, a transcriptome chip, a genome chip, a methylome chip, a metabolome chip, or the like.

The spatial omics chip of the embodiment of the present disclosure adopts a three-dimensional hydrophilic structure to form a hydrophilic region, and both the end face and the side surface of the hydrophilic structure can be connected to the coding probe, which can increase the contact area between the spatial omics chip and a test object, thereby improving the test efficiency. For example, the contact area between the spatial omics chip, and, for example, a tissue permeabilization product can be increased, thereby improving the capture efficiency of samples such as mRNA. In addition, the spatial omics chip of an embodiment of the present disclosure can increase a number of hydrophilic structures in the hydrophilic region by adjusting parameters such as a diameter and an arrangement of the hydrophilic structures in the hydrophilic region, thereby increasing a total surface area of the hydrophilic structures in the hydrophilic region and obtaining higher capture efficiency of the sample mRNA. In addition, each hydrophilic region is surrounded by the hydrophobic region, so that, on one hand, the tissue permeabilization product of the hydrophobic region does not enter into an inside of the hydrophilic region, and on the other hand, the mRNA sample captured by the hydrophilic region does not flow laterally, thereby reducing the cross-contamination between adjacent sample regions.

In an exemplary embodiment of the present disclosure, the hydrophilic structure can extend along a direction perpendicular to the first substrate.

In an exemplary embodiment of the present disclosure, each of the hydrophilic regions can contain a plurality of hydrophilic structures distributed in a periodic array. The periodic array distribution structure of a plurality of hydrophilic structures in the hydrophilic region can effectively increase the contact area between the hydrophilic region and the tissue permeabilization product, thereby improving the capture efficiency of the sample mRNA.

In an exemplary embodiment of the present disclosure, different hydrophilic structures all have a diameter of d₁, and a distance between two adjacent hydrophilic structures in each the hydrophilic region can be 0.25d₁ to 1.5d₁, for example, can be 0.25d₁, 0.5d₁, 0.75d₁, d₁, 1.25d₁, 1.5d₁.

In an exemplary embodiment of the present disclosure, a diameter d₁ of the hydrophilic structure can be 2 μm to 2.5 μm, and a height of the hydrophilic structure may be 5 μm to 10 μm.

In an exemplary embodiment of the present disclosure, as shown in FIG. 1A, the hydrophobic region 20 can contain a plurality of hydrophobic structures 21. The first substrate could have only one continuous hydrophobic region, and the one continuous hydrophobic region contains all of the hydrophobic structures; alternatively, as shown in FIG. 1A, the first substrate can have a plurality of hydrophobic regions connected together, wherein each of the hydrophobic regions surrounds one of the hydrophilic regions, and each of the hydrophobic regions contains a plurality of hydrophobic structures surrounding one of the hydrophilic regions.

In an exemplary embodiment of the present disclosure, different hydrophobic structures all have a diameter of d₂, and a distance between two adjacent hydrophobic structures can be 0.25d₂ to 1.5d₂.

In an exemplary embodiment of the present disclosure, d₂ can be 2 μm to 2.5 μm.

In an exemplary embodiment of the present disclosure, the diameter d₂ of all the hydrophobic structures 21 is the same as the diameter d₁ of all the hydrophilic structures 31, and the distance between adjacent hydrophobic structures 21 and hydrophilic structures 31 can be 0.25d₁ to 1.5d₁.

In an exemplary embodiment of the present disclosure, as shown in FIG. 1A, a plurality of hydrophilic regions can be distributed on the first substrate in a periodic array, and the distance between the centers of two adjacent hydrophilic regions is L₁, that is, the length of the arrangement period of the hydrophilic regions, L₁ can be not less than 1.5d₁, for example, L₁ can be greater than 4 d₁, or for another example, L₁ can be 4.446d₁ to 9d₁. For another example, L₁ can be 4.446d₁ (the hydrophilic region can be a regular triangle), 4.5d₁ (the hydrophilic region can be a square), 6d₁ (the hydrophilic region can be a regular hexagon), or 9d₁ (the hydrophilic region can be a regular hexagon). In an exemplary embodiment of the present disclosure, at least one hydrophobic structure 21 can be arranged between two adjacent hydrophilic regions 30, that is, the minimum distance between the edges of two adjacent hydrophilic regions 30 can be 2d₁.

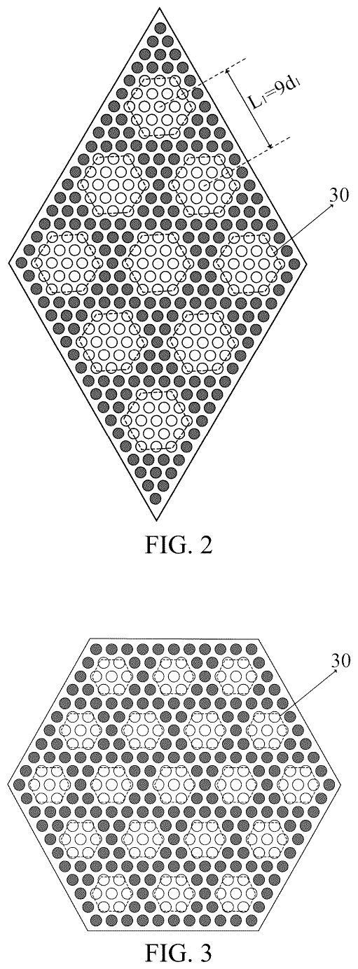

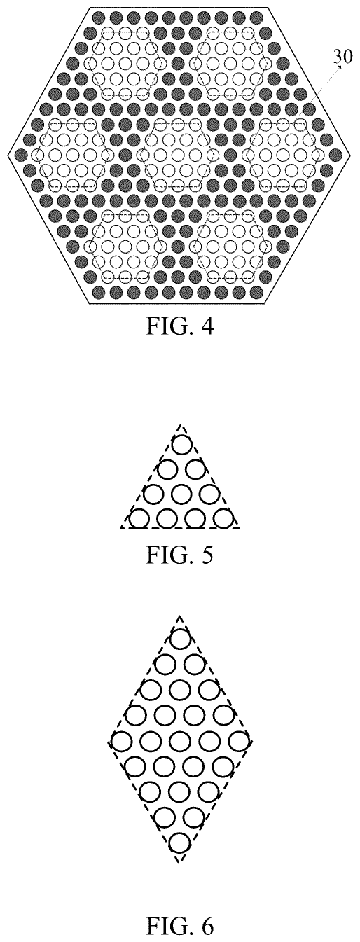

In an exemplary embodiment of the present disclosure, the hydrophilic structure and the hydrophobic structure can each independently contain any one or more of three-dimensional structures such as pillars, protrusions, and recesses. FIG. 2 is a top view of another spatial omics chip provided in an exemplary embodiment of the present disclosure; FIG. 3 is a top view of yet another spatial omics chip provided in an exemplary embodiment of the present disclosure; FIG. 4 is a top view of yet another spatial omics chip provided in an exemplary embodiment of the present disclosure.

As shown in FIG. 1A and FIGS. 2 to 4, the first substrate can be in a shape of a diamond, a regular polygon, for example, a regular hexagon; In other exemplary embodiments, the first substrate can also be in a shape of other polygons, for example, a regular triangle, a rectangle, a square, or the like, and can also be a circular or irregular shape.

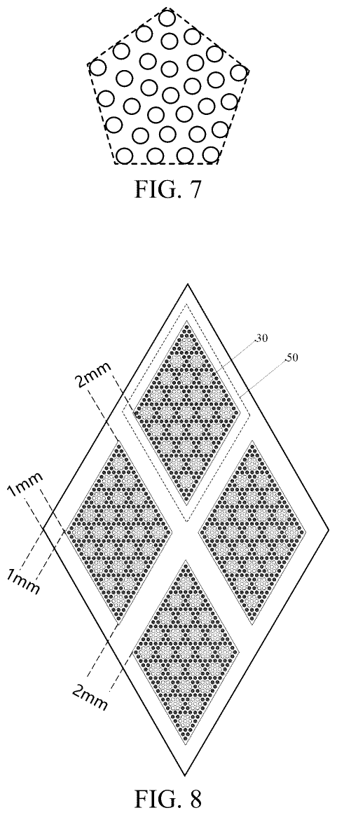

In an exemplary embodiment of the present disclosure, the hydrophilic region can be in a shape of a regular polygon or a combination of regular polygons, for example, the hydrophilic region can be in a shape of a regular triangle, a square, a regular pentagon, a regular hexagon, or the like, or for another example, the hydrophilic region can be in a shape of a diamond formed by combining two regular triangles. In other embodiments, the hydrophilic region can also be in a shape of a diamond, an oblong, or be irregular-shaped. FIG. 5 is a schematic diagram of a structure of a hydrophilic region of a spatial omics chip provided in an exemplary embodiment of the present disclosure; FIG. 6 is a schematic diagram of a structure of a hydrophilic region of another spatial omics chip provided in an exemplary embodiment of the present disclosure; FIG. 7 is a schematic diagram of a structure of a hydrophilic region of yet another spatial omics chip provided in an exemplary embodiment of the present disclosure. In the spatial omics chips shown in FIGS. 5, 6 and 7, the hydrophilic regions are equilateral triangles (regular triangles), diamonds formed by two equilateral triangles, and regular pentagons, respectively.

As shown in FIG. 1A and FIGS. 2 to 4, there can be two or three hydrophilic pillars on each side of the regular polygonal hydrophilic region 30; and in other exemplary embodiments, there can be more hydrophilic pillars on each side of the regular polygonal hydrophilic region 30, e.g., 4 pillars, 5 pillars, etc.

In the spatial omics chip as shown in FIG. 1A, the four sides of the diamond-shaped first substrate 10 can all be 6 mm in length, and the minimum internal angle can be 60°. Seven adjacent hydrophilic pillars form a regular hexagonal hydrophilic region 30, all of which are arranged in a periodic array and have the same size and shape. All hydrophobic pillars and all hydrophilic pillars have a diameter of d₁, and a distance between adjacent hydrophobic pillars and hydrophilic pillars, a distance between two adjacent hydrophobic pillars, and a distance between two adjacent hydrophilic pillars are 0.5d₁, so the length of the longest diagonal of each hydrophilic region is 4d₁, which is equivalent to the average size of mammalian cells, and can achieve single-cell level resolution. Each two adjacent hydrophilic regions 30 is separated by a hydrophobic pillar in a direction extending along the same diagonal line, that is, a minimum distance between the edges of the two adjacent hydrophilic regions 30 is 2d₁, and a distance L₁ between the centers of the two adjacent hydrophilic regions 30 is 6d₁, so that the hydrophilic regions 30 is distributed at a density of about 5132/mm² to 8018/mm² on the surface of the first substrate 10 at its detection resolution, and the detection resolution of the spatial omics chip is 12 μm.

In the spatial omics chips shown in FIGS. 2 and 4, the hydrophilic region 30 is in a regular hexagon shape, wherein each hydrophilic region 30 contains 19 hydrophilic pillars; all hydrophobic pillars and all hydrophilic pillars have a diameter of d₁; a distance between adjacent hydrophobic pillars and hydrophilic pillars, a distance between two adjacent hydrophobic pillars, and a distance between two adjacent hydrophilic pillars are 0.5d₁; and a distance L₁ between the centers of two adjacent hydrophilic regions 30 is 9d₁. If d₁ is 2 μm, a detection resolution of this spatial omics chip is 18 μm. In the hydrophilic region of the same size, the greater the number of hydrophilic pillars, the lower the detection resolution of the spatial omics chip, which can meet the testing requirements of lower resolution. In an exemplary embodiment of the present disclosure, the hydrophilic structure can be connected to the first substrate by a carbon-hydrogen bond.

In an exemplary embodiment of the present disclosure, the first substrate can be a silicon wafer, a glass wafer or a plastic wafer. Materials of the plastic wafer include, but are not limited to, polymethyl methacrylate (PMMA), polycarbonate (PC), and cyclic olefin copolymer (COC), etc. These materials can be surface modified and grafted to generate hydrophilic structures or hydrophobic structural arrays to meet subsequent analytical testing requirements for spatial omics such as spatial transcriptomics.

In an exemplary embodiment of the present disclosure, the surface of the hydrophilic structure contains an active group capable of being connected to the coding probe, and the active group is used for subsequent connection to the coding probe, thereby connecting the coding probe to the hydrophilic structure.

The active group can include any one or more of an amino group, a thiol group, a carboxyl group, an ester group and an anhydride; the hydrophobic structures each does not carry the active group, the hydrophobic structures each contains a hydrophobic group, and the hydrophobic group contains any one or more of a long-chain hydrocarbon group having a carbon chain length ≥7 and a fluoroalkyl group.

In an exemplary embodiment of the present disclosure, the spatial omics chip can further include a coding probe connected to a hydrophilic structure; and the coding probe includes a sequencing primer binding sequence, a spatial position tag sequence, a UMI sequence and a Poly(dT) sequence.

In an exemplary embodiment of the present disclosure, the sequencing primer binding sequence, the spatial position tag sequence, the UMI sequence, and the Poly(dT) sequence are sequentially connected.

Along a direction away from the hydrophilic structure, the coding probe sequentially includes a sequencing primer binding sequence (Partial Read 1), a spatial position tag sequence (Spatial Barcode), a UMI sequence and a Poly(dT) sequence.

The sequencing primer binding sequence is used for complementary pairing with a primer during on-machine sequencing, and the sequencing primer binding sequences are the same for all the coding probes connected to the hydrophilic structures on the first substrate.

The spatial position tag sequence is used to mark a specific spatial position of a subsequently captured mRNA sample. The spatial position tag sequences are the same for the coding probes connected to the hydrophilic structures located in same the hydrophilic region, indicating that mRNA samples with the same nucleic acid sequence structure are derived from the same cell. The spatial position tag sequences are different for the coding probes connected to the hydrophilic structures located in different hydrophilic regions, indicating that the mRNA samples with different nucleic acid sequence structures are derived from different cells, thereby distinguishing the spatial positions of different cells. Therefore, the spatial position tag sequence is a segment of sequence of particular importance in the process of spatial transcriptome sequencing analysis.

The UMI sequence is used to specifically mark different mRNA samples within the same cell in order to quantify all different mRNA samples, and the UMI sequence are different for all the coding probes connected to the hydrophilic structures on the first substrate.

The Poly(dT) sequence is used to capture the mRNA sample and is a segment of sequence consisting of contiguous T bases.

In an exemplary embodiment of the present disclosure, the coding probe is connected to both the end face and the side surface of the hydrophilic structure, which is beneficial to improve the capture efficiency of the mRNA sample.

In an exemplary embodiment of the present disclosure, one end (typically the 5′ end) of the sequencing primer binding sequence connected to the hydrophilic structure carries a linking group, which can include a carboxyl group, a thiol group and an amino group. The linking group is used to react with an active group on the hydrophilic structure, thereby achieving connection of the sequencing primer binding sequence to the hydrophilic structure.

In an exemplary embodiment of the present disclosure, the sequencing primer binding sequence and the hydrophilic structure can be connected together by at least one of an amino-carboxyl reaction, a thiol-thiol reaction, and an anhydride-amino reaction.

In an exemplary embodiment of the present disclosure, the sequencing primer binding sequence and the hydrophilic structure can be connected together by at least one of an amide group and a disulfide bond. For example, the active group on the hydrophilic structure can be an amino group, the linking group on the sequencing primer binding sequence is a carboxyl group, and the sequencing primer binding sequence and the hydrophilic structure can be connected by an amide group formed by the reaction of the amino group with the carboxyl group.

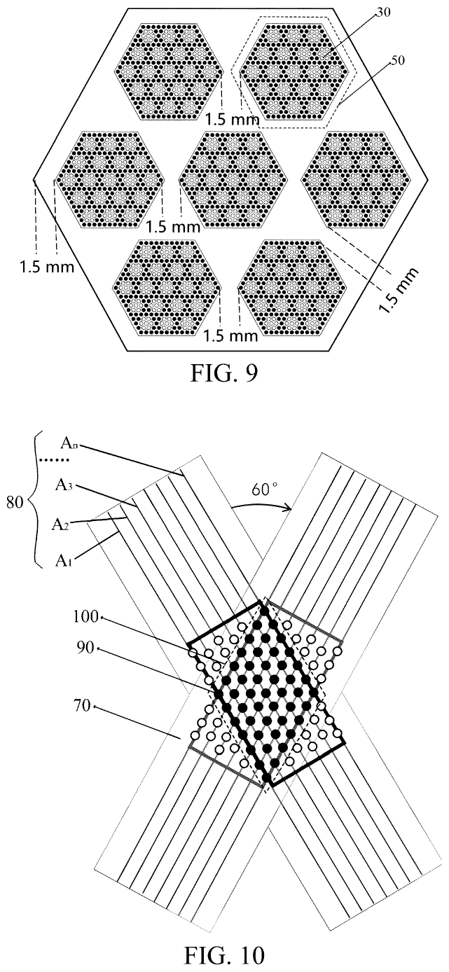

FIG. 8 is a top view of yet another spatial omics chip provided in an exemplary embodiment of the present disclosure; FIG. 9 is a top view of yet another spatial omics chip provided in an exemplary embodiment of the present disclosure.

In an exemplary embodiment of the present disclosure, as shown in FIGS. 8 and 9, the spatial omics chip can contain a plurality of sub-chips 50 distributed at intervals, the plurality of sub-chips 50 are distributed on the first substrate 10 in a periodic array, and the sub-chip 50 contain a plurality of hydrophilic regions 30.

The spatial omics chip shown in FIG. 8 integrates four diamond-shaped sub-chips 50 on a diamond-shaped first substrate, and the spatial omics chip shown is a 4-flux spatial omics chip. In an extension direction of the side of the diamond-shaped sub-chip 50, a distance between two adjacent sub-chips 50 can be 2 mm, and a distance between the sub-chip 50 and the edge of the first substrate can be 1 mm.

The spatial omics chip shown in FIG. 9 integrates seven regular hexagonal sub-chips 50 on a regular hexagonal first substrate, and the spatial omics chip shown is a 7-flux spatial omics chip; and a minimum distance between two adjacent sub-chips 50 can be 1.5 mm.

An embodiment of the present disclosure also provides a rotary step-by-step sample injection chip configured to be capable of perform step-by-step sample injection into a hydrophilic structure of the hydrophilic region of the spatial omics chip as provided in an embodiment of the present disclosure above.

In the description of the present disclosure, “step-by-step sample injection” refers to multiple (at least two) sample injections.

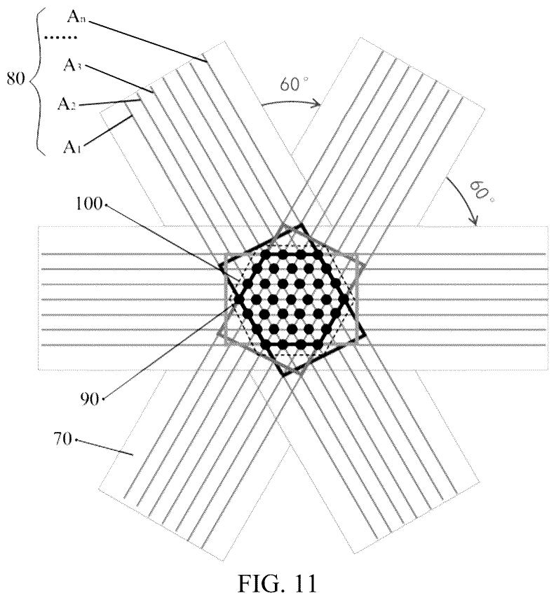

FIG. 10 is a top view of a rotary step-by-step sample injection chip provide by an exemplary embodiment of the present disclosure; FIG. 11 is a top view of another rotary step-by-step sample injection chip provided in an exemplary embodiment of the present disclosure.

As shown in FIGS. 10 and 11, the rotary step-by-step sample injection chip contains a second substrate 70 and a plurality of flow channels 80 arranged on the same side of the second substrate 70 and distributed at intervals, including a first flow channel A1, a second flow channel A2, a third flow channel A3, . . . , and an n-th flow channel An sequentially. The flow channel 80 has a plurality of through holes 90 that penetrated the second substrate 70 and are distributed in a periodic array. Each hydrophilic region 30 of the spatial omics chip corresponds to a through hole 90, and a distance between the centers of two adjacent through holes 90 is the same as the distance between the centers of two adjacent hydrophilic regions 30 corresponding to the two through holes 90. The through holes 90 are configured to perform step-by-step sample injection into the hydrophilic regions 30 corresponding thereto.

In an exemplary embodiment of the present disclosure, the through hole is configured to perform sample injection into only the hydrophilic region corresponding thereto and not into the hydrophobic region.

In an exemplary embodiment of the present disclosure, the rotary step-by-step sample injection chip can contain an effective sample injection region 100. The rotary step-by-step sample injection chip is rotated around a center thereof at least once at a set angle, and at least part of the through holes of the rotary step-by-step sample injection chip before and after at least one rotation can be completely overlapped, therefore the through holes that can be completely overlapped form the effective sample injection region 100.

In the description of the present disclosure, “the through holes being completely overlapped” refers the through holes before and after rotation have a common overlapping region. For example, a center of the through hole after rotation is the same as that of the through hole before rotation.

In an exemplary embodiment of the present disclosure, when the spatial omics chip is matched with the rotary step-by-step sample injection chip, the orthographic projection of the through holes in the effective sample injection region on the second substrate is overlapped with the hydrophilic region of the spatial omics chip on the second substrate, which can be completely overlapped or cross-overlapped areas. If the spatial omics chip contains sub-chips, the effective sample injection regions 100 can correspond to the sub-chips of the spatial omics chip, that is, the effective sample injection regions 100 and the sub-chips have the same shape and size, and the orthographic projection of the through hole in the effective sample injection region on the second substrate is overlapped with the orthographic projection of the hydrophilic region of the sub-chip on the second substrate. If the spatial omics chip does not contain sub-chips, the effective sample injection region 100 can correspond to the spatial omics chip, that is, the effective sample injection region 100 and the spatial omics chip have the same shape and size, and the orthographic projection of the through hole in the effective sample injection region on the second substrate is overlapped with the orthographic projection of the hydrophilic region of the spatial omics chip on the second substrate.

In an exemplary embodiment of the present disclosure, the set angle of rotation of the rotary step-by-step sample injection chip can be set according to the desired shape of the effective sample injection region, for example, a minimum inner angle of a diamond, a center angle of a regular polygon, or the like, or for another example, 30°, 45°, 60°, 90°, or the like.

In an exemplary embodiment of the present disclosure, the effective sample injection region can be a regular polygon having n sides, and n is an integer ≥3;
a distance between the centerlines of two adjacent flow channels satisfies:

$${L}_{3} = {L}_{2} \times sin θ ;$$

wherein L₃ is a distance between two adjacent flow channels;
L₂ is a distance between the centers of two adjacent through holes; and
θ is a rotation angle of the rotary step-by-step sample injection chip.

For example, if the effective sample injection region 100 is square, the effective sample injection region 100 is obtained by rotating the rotary step-by-step sample injection chip once at a rotation angle of 90°.

For example, if the effective sample injection region 100 is a regular polygon having n sides, and n is an even number, the effective sample injection region 100 is obtained by rotating the rotary step-by-step sample injection chip n/2 times at a rotation angle of the central angle θ of the regular polygon.

In an exemplary embodiment of the present disclosure, the second substrate can be a silicon wafer or a glass wafer, and can be in a rectangle or other shape. The flow channels can be parallel to each other and extend along a direction parallel to a long side of the rectangular second substrate. The diameter of the hydrophilic structure is d₁, the length of the second substrate can be 3×749×6×d₁, the width can be 499×5.196×d₁, the length and width both take an integer of millimeters (mm) upward, and the thickness is 2 mm.

When all hydrophilic structures of the spatial omics chip have a diameter d₁ of 2 μm, the rectangular second substrate can have 500 flow channels evenly distributed in the extension direction of its short side, and a distance between the center lines of two adjacent flow channels is 5.196×d₁ (i.e., the arrangement period length of the hydrophilic region×sin 30°, d₁ is the diameter of the hydrophilic structure). A length of each flow channel is the length of the second substrate. The through holes are distributed in the middle ⅓ of the flow channel (in the rectangular box in FIG. 10). A number of through holes on a flow channel is at an alternating cycle of 749 and 750. For example, a first flow channel A1 is 750 (or 749), a second flow channel A2 is 749 (or 750), a third flow channel A3 is 750 (or 749), a fourth flow channel is 749 (or 750) . . . and so on, the sample can be injected downward through these through holes when flowing in the flow channel. The through-hole 90 located in the effective sample injection region 100 exactly corresponds to a hydrophilic region on the surface of the substrate of the spatial omics chip, so all samples passing through the through-hole can enter a unique hydrophilic region, that is, the distance between two adjacent through-holes on the same channel is the same as the arrangement period length between the hydrophilic regions of the spatial omics chip provided in an embodiment of the present disclosure, which is 6d₁.

In an exemplary embodiment of the present disclosure, a diameter of the through hole is slightly smaller than a diameter of the hydrophilic region. For example, if the hydrophilic region is a diamond formed by 7 hydrophilic structures, and a diameter of the hydrophilic region is 4d₁, the diameter of the hole can be 3×d₁ to ensure the accuracy of all sample injections.

In an exemplary embodiment of the present disclosure, an area of the through hole can be less than or equal to an area of the hydrophilic region corresponding thereto.

An embodiment of the present disclosure also provides a synthesis method for a spatial position tag sequence, including:
injecting a sub-spatial position tag sequence multiple times via a through hole of the rotary step-by-step sample injection chip as provided in an embodiment of the present disclosure above, and rotating the rotary step-by-step sample injection chip around a center thereof between two adjacent injections at a set angle, which causes at least part of the through holes of the rotary step-by-step sample injection chip before and after the rotation to completely overlap, and injecting the sub-spatial position tag sequence into a hydrophilic region of the spatial omics chip via the through holes of the overlapping region to form the spatial position tag sequence;
wherein the sub-spatial position tag sequences injected into the plurality of through holes on the same flow channel are the same, and the sub-spatial position tag sequences injected into the through holes on different flow channels are different.

The synthesis method for a spatial position tag sequence according to an embodiment of the present disclosure can adopt sub-spatial position tag sequences whose sequence are known and perform sample injection multiple times, which connects the sub-spatial position tag sequences to form the spatial position tag sequence. Therefore, the sequence of the synthesized spatial position tag sequence is also known. When a spatial omics analysis is performed by using the spatial omics chip connected to the spatial position tag sequence, there is no need to sequence and decode spatial position tag information in advance by a method such as complex in-situ sequencing. This can effectively simplify the spatial transcriptome analysis process and reduce the detection cost.

In an exemplary embodiment of the present disclosure, injecting the sub-spatial position tag sequence into a hydrophilic region of the spatial omics chip via the through holes of the overlapping region can include:
bonding the rotary step-by-step sample injection chip to the spatial omics chip as provided above in an embodiment of the present disclosure, such that a hydrophilic region of the spatial omics chip corresponds to a through hole of a flow channel of the rotary step-by-step sample injection chip, and the hydrophilic structure of the hydrophilic region is connected to a nucleic acid sequence (e.g., a sequencing primer binding sequence of the coding probe); and
injecting different sub-spatial position tag sequences into different flow channels of the rotary step-by-step sample injection chip respectively, wherein the sub-spatial position tag sequences enter the hydrophilic region of the spatial omics chip via the through hole on the flow channel, and are connected to the nucleic acid sequences connected on the hydrophilic structure (e.g., to the sequencing primer binding sequence, or to a sub-spatial position tag sequence to which the sequencing primer binding sequence is connected).

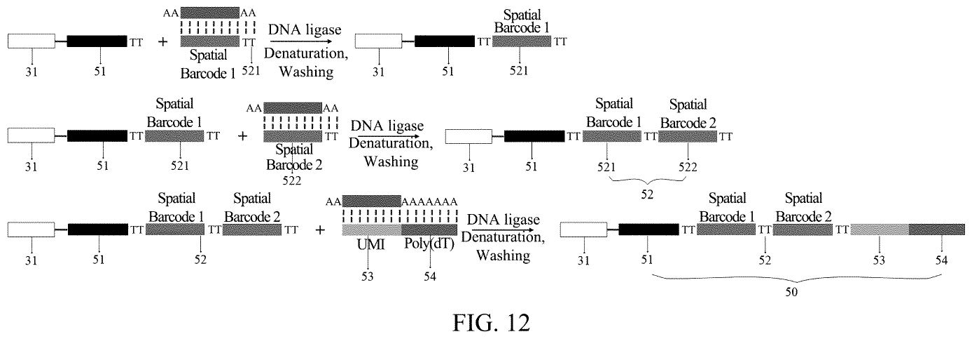

In an exemplary embodiment, the spatial position tag sequence can be divided into two segments for connection, wherein a first segment is a first sub-spatial position tag sequence 521 (Spatial Barcode 1) and a second segment is a second sub-spatial position tag sequence 522 (Spatial Barcode 2). FIG. 12 is a process flow diagram of a method of forming a coding sequence provided in an exemplary embodiment of the present disclosure. As shown in FIG. 12, after the sequencing primer binding sequence 51 is connected to the hydrophilic structure 31, the first sub-spatial position tag sequence 521 (Spatial Barcode 1) is first connected at the 3′ end of the sequencing primer binding sequence 51 through an adapter structure of Spatial Barcode 1 under an action of DNA ligase, then the second sub-spatial position tag sequence 522 (Spatial Barcode 2) is connected at the 3′ end of the first sub-spatial position tag sequence 521 through an adapter structure of Spatial Barcode 2 also under an action of DNA ligase, thereby forming a complete spatial position tag sequence 52. The UMI sequence 53 and the Poly(dT) sequence 54 are connected at the end of the spatial position tag sequence 52 by means of a combined adapter structure formed by the ligation of the UMI sequence 53 and the Poly(dT) sequence 54, forming a complete coding probe sequence. In the above synthesis process, the first sub-spatial position tag sequence 521 and the second sub-spatial position tag sequence 522 of the spatial position tag sequence 52 are actually the same set of sequences, and the purpose of describing the first sub-spatial position tag sequence 521 and the second sub-spatial position tag sequence 522 separately here is only to distinguish the sample injection order, and the specific method involved in this sample injection order is a rotary step-by-step sample injection method.

The rotary step-by-step sample injection achieves a connection of a first sub-spatial position tag sequence using a rotary step-by-step sample injection chip at a first sample injection, thereafter rotates the rotary step-by-step sample injection chip around a center thereof at a set angle (for example, rotating 60° clockwise), and then achieves a connection of the second sub-spatial position tag sequence through a second sample injection. Thus a spatial position tag sequence is synthesized by two sample intakes and connection.

Next, the synthesis process of spatial position tag sequence is illustrated, taking a step-by-step sample injection from the rotary step-by-step sample injection chip as shown in FIG. 10 into the spatial omics chips as shown in FIG. 1A or FIG. 1B as an example. It should be emphasized that when the rotary step-by-step sample injection chip is used, it is needed to closely cover the surface on the side of the spatial omics chip arranged with the hydrophilic region provided in an embodiment of the present disclosure, so as to successfully complete the sample injection. Furthermore, each hydrophilic region is made to correspond to one through hole, so that the through hole can perform sample injection into only the hydrophilic region corresponding thereto, and cannot perform sample injection into the hydrophilic region not corresponding thereto. For example, each of the hydrophilic regions can be located below and communicate with one of the through holes, and the projection of each of the through holes on the first substrate can fall within the range of the projection of the hydrophilic region corresponding to the through hole on the first substrate, or even the center of the projection of each of the through holes on the first substrate can be overlapped with the center of the projection of the hydrophilic region corresponding to the through hole on the first substrate. Assume that there are 500 flow channels on the rotary step-by-step sample injection chip, and the first sub-spatial position tag sequences (including the first tag sequence B₁, the first tag sequence B₂, the first tag sequence B₃, . . . , the first tag sequence B₅₀₀) with known sequences are respectively flowed into different flow channels during the first sample injection, and such 500 first tag sequences are all different from each other. After the DNA polymerase ligation reaction is completed, the rotary step-by-step sample injection chip is rotated 60° clockwise around its center, and then a second sample injection is performed. At this time, 500 flow channels continued to flow into 500 second sub-spatial position tag sequences (including the second tag sequence C₁, the second tag sequence C₂, the second tag sequence C₃, . . . , the second tag sequence C₅₀₀) whose sequences are known and different from each other.

It should be emphasized that the second sub-spatial position tag sequences flowed into each flow channel after rotation are respectively the same as the first sub-spatial position tag sequences flowed into the flow channel before rotation, that is, the second tag sequence C1 is the same as the first tag sequence B1, the second tag sequence C2 is the same as the first tag sequence B2, the second tag sequence C3 is the same as the first tag sequence B3, . . . , the second tag sequence C500 is the same as the first tag sequence B500, which is equivalent to 500 known tag sequences different from each other being introduced twice before and after rotation, and performing two ligation reactions. The first tag sequence and the second tag sequence (or the first sub-spatial position tag sequence and the second sub-spatial position tag sequence) are used here only to distinguish the order of sample injection. The rotary step-by-step sample injection chip completes sample injection twice before and after rotation, and the hydrophilic regions corresponding to and below the positions where the sample injection through holes intersects sequentially achieves the ligation of the first tag sequence and the second tag sequence, that is, the ligation and synthesis of the spatial position tag sequence are achieved on each hydrophilic region of the spatial omics chip. Subsequently, the UMI sequence and the Poly(dT) sequence are sequentially connected to the spatial position tag sequence, thus obtaining a spatial omics chip with a coding probe connected to the hydrophilic structure. This chip has both mRNA sample capture function and sample spatial position marking function, which can meet the needs of subsequent spatial transcriptome sequencing and analysis.

In the synthesis method for a spatial position tag sequence by the rotary step-by-step sample injection mentioned above, if the first substrate of the spatial omics chip is a diamond with a side length of 6 mm and a minimum internal angle of 60°, the diameters d₁ of the hydrophilic structure and d₂ of the hydrophobic structure are both 2 μm, and there are 500 flow channels on the rotary step-by-step sample injection chip, then 250,000 (500×500) different coding probes can be combined by permutations. In theory, 250,000 cell samples can be detected simultaneously with a detection resolution of 12 μm. Moreover, since the first tag sequence and the second tag sequence are both known sequences, the spatial position tag sequence formed by combining the two sequences by permutations. is also known, spatial omics analysis using the spatial omics chip connected with the spatial position tag sequence does not need to sequence and decode spatial position tag information in advance by complex in-situ sequencing and other methods, and can effectively simplify the spatial transcriptome analysis process and reduce the detection cost.

In an exemplary embodiment of the present disclosure, the spatial position tag sequence with higher discrimination and accuracy can be obtained by adjusting the number of step-by-step sample injection (or the number of rotations of the rotary step-by-step sample injection chip), and the erroneous analysis results caused by the spatial position tag sequence test error or the unexpected identity of the sequence combination in the library construction and sequencing process can be more effectively avoided, so that the sample test requirements with higher precision and accuracy can be met.

Exemplarily, the number of step-by-step sample injection can be three, and at this time, the number of rotations of the rotary step-by-step sample injection chip is two, and the shape of the spatial omics chip caused by the adjustment method is changed, so that the spatial omics chip as shown in FIG. 3 can be obtained. As shown in FIG. 3, the effective sample injection region formed after two rotations of the rotary step-by-step sample injection chip at an angle of 60° can be a regular hexagon. Compared with the spatial omics chip shown in FIG. 1A obtained by one rotation, the shape, size and spacing of the hydrophilic regions formed by the hydrophilic structure does not change. When the diameter d₁ of the hydrophilic structure is 2 μm, the detection resolution of the spatial omics chip shown in FIG. 3 does not change compared with the spatial omics chip shown in FIG. 1A, and is 12 μm. However, it is noted that at this time, the number of flow channels of the rotary step-by-step sample injection chip can only be set to an odd number, because when the number of sample injection is odd, the number of flow channels of the rotary step-by-step sample injection chip should also be odd number; and when the number of sample injection is even, the number of flow channels of the rotary step-by-step sample injection chip should also be even. If the number of sample injection is not matched with the number of flow channels of the rotary step-by-step sample injection chip according to the above rules, the through holes on each flow channel after the latter rotation will not fall accurately at the intersection position of the two flow channels formed after the previous rotation, which will lead to the failure of the latter sample injection. In addition, the number and position distribution of through holes on each flow channel also need to be adjusted accordingly, and the structure of photomask related to the synthesis of hydrophilic structure and hydrophobic structure should also change accordingly.

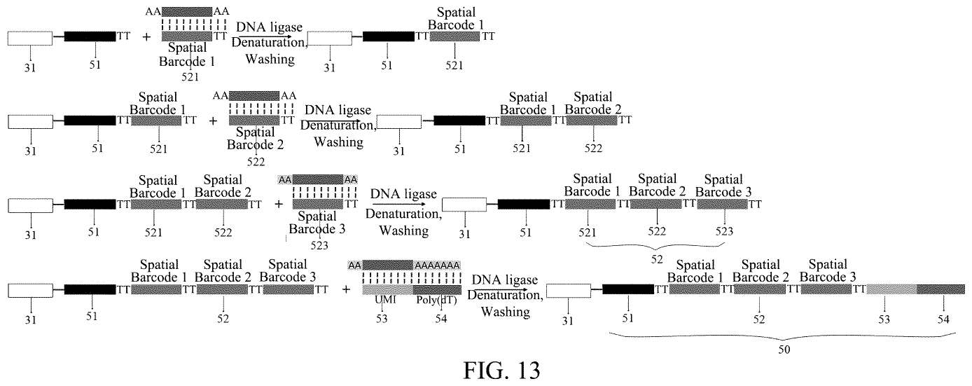

Since the number of sample injection have changed from one to three, the synthesis method for the coding probe sequence is also different from the process flow shown in FIG. 12. FIG. 13 is a process flow diagram of another method of forming a coding sequence provided in an exemplary embodiment of the present disclosure. As shown in FIG. 13, the spatial position tag sequence coding the probe sequence is divided into the first sub-spatial position tag sequence 521 (Spatial Barcode 1), the second sub-spatial position tag sequence 522 (Spatial Barcode 2), and the third sub-spatial position tag sequence 523 (Spatial Barcode 3), which are sequentially connected, and there is no significant change in the linkage of other parts.

An embodiment of the present disclosure also provided a manufacturing method of a spatial omics chip as provided in an embodiment of the present disclosure above, including:
forming a hydrophilic region containing a hydrophilic structure on a first substrate;
forming a hydrophobic region entirely surrounding the hydrophilic region on the first substrate.

In an exemplary embodiment of the present disclosure, forming a hydrophilic region containing a hydrophilic structure on the first substrate can include:
covering the first substrate with a first photomask having a first light-transmitting region and a first light-shielding region, wherein the first light-transmitting region completely coincided with an orthographic projection of a region on the first substrate where the hydrophilic structure is to be formed on the first substrate; and
under illumination conditions, using a hydrophilic monomer as a raw material, performing a graft polymerization reaction on a region on the first substrate that completely coincides with the orthographic projection of the first light-transmitting region to form a hydrophilic polymer, and wherein the hydrophilic polymer contains an active group capable of being connected to the coding probe, and the hydrophilic polymer forms the hydrophilic structure.

In an exemplary embodiment of the present disclosure, forming a hydrophobic region entirely surrounding the hydrophilic region on the first substrate can include:
covering the first substrate with a second photomask having a second light-transmitting region and a second light-shielding region, wherein the second light-transmitting region completely coincides with an orthographic projection of a region on the first substrate where the hydrophobic structure is to be formed on the first substrate; and
under illumination conditions, using a hydrophobic monomer as a raw material, performing a graft polymerization reaction on a region on the first substrate that completely coincides with the orthographic projection of the second light-transmitting region to form a hydrophobic polymer, wherein the hydrophobic polymer contains a hydrophobic group, the hydrophobic polymer forms a hydrophobic structure, and the hydrophobic structure forms the hydrophobic region.

In the manufacturing method of the spatial omics chip provided in the exemplary embodiment of the present disclosure described above, the hydrophilic structure and the hydrophobic structure are formed on the surface of the first substrate under irradiation of ultraviolet light or visible light by means of two photomasks having complementary light-transmitting structures, respectively, and by using a hydrophilic monomer and a hydrophobic monomer as synthetic raw materials, respectively. Such manufacturing method is efficient and simple, and the conditions are mild and controllable.

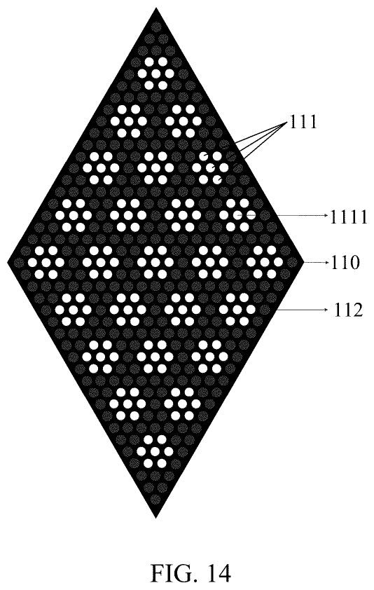

FIG. 14 is a top view of a first photomask provided in an exemplary embodiment of the present disclosure. As shown in FIG. 14, the first photomask has a first light-transmitting region 111 and a first light-shielding region 112 arranged on the first background layer 110. Exemplarily, the first light-transmitting region 111 can contain a plurality of first light-transmitting holes 1111 penetrating the first background layer 110, and the projection of the first light-transmitting holes 1111 on the first substrate can completely overlap with the projection of the hydrophilic structure on the first substrate. The first light-transmitting region 111 is a position where the hydrophilic structure is located, and the position where the hydrophobic structure is located is located on the first light-shielding region 112, both of which are in a light-shielding state.

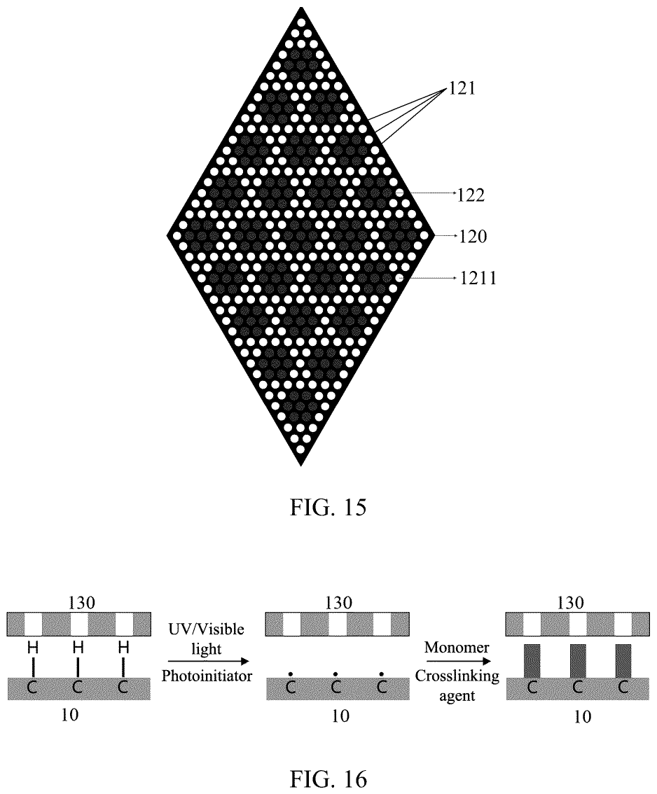

FIG. 15 is a top view of a second photomask provided in an exemplary embodiment of the present disclosure. As shown in FIG. 15, the second photomask has a second light-transmitting region 121 and a second light-shielding region 122 arranged on the second background layer 120. Exemplarily, the second light-transmitting region 121 can contain a plurality of second light-transmitting holes 1211 penetrating the second background layer 120, and the projection of the second light-transmitting holes 1211 on the second substrate can completely overlap with the projection of the hydrophobic structure on the second substrate. The second light-transmitting region 121 is a position where the hydrophobic structure is located, and the position where the hydrophilic structure is located is located on the second light-shielding region 122, both of which are in a light-shielding state.

In an exemplary embodiment of the present disclosure, the shape and size of the first photomask and the second photomask can be the same as the shape and size of the first substrate, the size and distribution spacing of the first light-transmitting hole can be the same as the size and distribution spacing of the hydrophilic structure on the surface of the first substrate, and the size and distribution spacing of the second light-transmitting hole can be the same as the size and distribution spacing of the hydrophobic structure on the surface of the first substrate.

In an exemplary embodiment of the present disclosure, when the first substrate is a silicon wafer or a glass wafer, the manufacturing method for the spatial omics chip can further include: modifying the first substrate with a silane coupling agent having a saturated or unsaturated hydrocarbon group before forming a hydrophilic region and a hydrophobic region on the surface of the first substrate, so that a carbon-hydrogen bond (C—H) is introduced on the surface of the first substrate.

In an exemplary embodiment of the present disclosure, the silane coupling agent can include, but is not limited to, methyltriethoxysilane, octadecyltrimethoxysilane, γ-methacryloxypropyltriethoxysilane, vinyltrimethoxysilane, and the like.

When the first substrate is a plastic wafer of such as polymethyl methacrylate, polycarbonate, or cycloolefin copolymer, there is no need to modify the first substrate.

In the above manufacturing method, the growth and formation of the hydrophilic structure and the hydrophobic structure on the surface of the first substrate mainly utilizes the principle of ultraviolet/visible light-induced graft polymerization on the surface of the first substrate material containing C—H. FIG. 16 is a reaction mechanism diagram of the graft polymerization reaction on the surface of the first substrate.

As shown in FIG. 16, the photoinitiator captures H atoms on the surface of the first substrate 10 containing C—H under the excitation of ultraviolet/visible light, which forms a large number of active radicals on the surface of the first substrate 10 in turn. These active radicals can further initiate the graft polymerization reaction of hydrophilic monomers or hydrophobic monomers in the reaction system on the surface of the first substrate 10, and with the assistance of the two photomasks 130 above, the hydrophilic structure or hydrophobic structure can be formed on the surface of the first substrate 10 after the graft polymerization is finally completed. The composition of the reaction system for preparing the hydrophilic structure and the hydrophobic structure is the same except the reaction monomers, which are different. The other components include solvents (including but not limited to water, ketones such as acetone, alcohols such as ethanol, esters such as ethyl acetate, ethers such as dioxane, and amides such as N, N-dimethylformamide), initiators (including but not limited to benzophenone and its derivatives, thioxanthones, anthraquinones) and crosslinking agents (including but not limited to unsaturated dienes such as divinylbenzene, acrylates such as polyethylene glycol dimethacrylate), etc.

In an exemplary embodiment of the present disclosure, the hydrophilic monomer can have an olefin group and a hydrophilic group, and the hydrophilic group can include any one or more of an amino group, a carboxyl group, and an ester group. For example, the hydrophilic monomers include, but are not limited to: acrylic acid, mercapto polyethylene glycol acrylate, vinyl acetate, 2-aminoethyl methacrylate. Such monomers, after homopolymerization to form a hydrophilic structure, still retained reactive groups having subsequent reactivity with the coding probe on the surface of the hydrophilic structure.

The hydrophilic monomer can also contain only a hydrophilic structure and an alkene group, and itself does not contain an active group having subsequent reactivity with the coding probe, for example, polyethylene glycol methacrylate or the like. However, such monomers need to be copolymerized with other olefins with reactive groups (such as maleic anhydride, etc.) to ensure that the surface of the hydrophilic structure formed after polymerization can retain reactive groups having subsequent reactivity with the coding probe.

In an exemplary embodiment of the present disclosure, the hydrophobic monomer can have an olefin group and a hydrophobic group, and the hydrophobic group can include any one or more of a long-chain hydrocarbon group and a fluoroalkyl group. The carbon chain length of the long chain hydrocarbon group can be ≥7, for example, can be 8 to 20, and can also be 12 to 18. For example, the hydrophobic monomer includes, but is not limited to, dodecyl methacrylate, heptadecyl methacrylate, trifluoroethyl acrylate, perfluoroalkyl ethyl acrylate, dodecafluoroheptyl methacrylate, and the like. The hydrophobic monomer can also be copolymerized with other unsaturated olefins, and it should be emphasized that neither the hydrophobic monomer nor the monomer copolymerized therewith can have any active groups having subsequent reactivity with the coding probe after polymerization.

In an exemplary embodiment of the present disclosure, the manufacturing method of a spatial omics chip can further include sequentially ligating a sequencing primer binding sequence, a spatial position tag sequence, a UMI sequence and a Poly(dT) sequence at an end of the hydrophilic structure away from the first substrate.

In an exemplary embodiment of the present disclosure, the spatial position tag sequence can be formed by a synthesis method of the spatial position tag sequence as provided in an embodiment of the present disclosure above.

In order to adapt to the detection requirements of different resolutions, the detection resolution and accuracy can be adjusted by designing different photomask structures to adapt to different sample detection requirements. The accuracy of spatial positioning of mRNA samples can also be increased by adjusting the number of rotary step-by-step sample injection during spatial position tag sequence synthesis to meet the sample detection requirements with higher accuracy.

When the number of flow channels of the rotary step-by-step sample injection chip and the size, number and position distribution of through holes on the flow channels for sample injection need to be adjusted, the distribution of light-transmitting holes of the photomask related to the synthesis of hydrophilic structure and hydrophobic structure should also change accordingly.

Although implementation modes of the present disclosure are disclosed above, contents described are only implementation modes used for ease of understanding of the present disclosure, but not intended to limit the present disclosure. Those skilled in the art may make any modification and change in the forms and details of the implementations without departing from the essence and scope of the present disclosure. However, the scope of protection of the present disclosure should still be subject to the scope defined by the attached claims.

1. A spatial omics chip, comprising: a first substrate, hydrophobic regions arranged on the same side of the first substrate, and a plurality of hydrophilic regions distributed in a periodic array and spaced apart from each other, wherein each hydrophilic region is surrounded by the hydrophobic regions, and the hydrophilic region comprises a hydrophilic structure configured to be connected to a coding probe.

2. The spatial omics chip according to claim 1, wherein each of the hydrophilic regions comprises a plurality of the hydrophilic structures distributed in a periodic array, wherein different hydrophilic structures all have a diameter of d₁, and a distance between two adjacent hydrophilic structures in each of the hydrophilic regions is 0.25d₁ to 1.5d₁.

3. The spatial omics chip according to claim 2, wherein a diameter d₁ of the hydrophilic structure is 2 μm to 2.5 μm, and a height of the hydrophilic structure is 5 μm to 10 μm.

4. The spatial omics chip according to claim 2, wherein the hydrophobic region comprise a plurality of hydrophobic structures, and at least one hydrophobic structure is arranged between two adjacent hydrophilic regions; wherein different hydrophobic structures all have a diameter of d₂, and a distance between two adjacent hydrophobic structures is 0.25d₂ to 1.5d₂, d₂ is 2 μm to 2.5 μm; and
a distance between adjacent hydrophobic structures and hydrophilic structures is d₃.

5. The spatial omics chip according to claim 4, wherein d₁=d₂ and d₃ is 0.25d₁ to 1.5d₁.

6. The spatial omics chip according to claim 4, wherein the plurality of hydrophilic regions are distributed on the first substrate in a periodic array, and a distance between centers of two adjacent hydrophilic regions is L₁, and L₁ is not less than 1.5d₁.

7. The spatial omics chip according to claim 6, wherein L₁ is 4.446d₁ to 9d₁.

8. The spatial omics chip according to claim 1, wherein the hydrophilic structure is connected to the first substrate by a carbon-hydrogen bond; a surface of the hydrophilic structure comprises an active group capable of being connected to the coding probe, and the active group comprises any one or more of an amino group, a thiol group, a carboxyl group, an ester group and an anhydride; wherein each of the hydrophobic structures comprises thereon a hydrophobic group comprising any one or more of a long-chain hydrocarbon group having a carbon chain length ≥7 and a fluoroalkyl group.

9. The spatial omics chip of claim 8, further comprising the coding probe connected to the hydrophilic structure; wherein the coding probe comprises any one or more of a chemical probe, a polypeptide probe, a fluorescent probe, and a nucleic acid probe.

10. The spatial omics chip of claim 9, wherein the coding probe is configured to capture an mRNA sample and mark a spatial position of the mRNA sample captured.

11. The spatial omics chip of claim 10, wherein the coding probe comprises a sequencing primer binding sequence, a spatial position tag sequence, a UMI sequence and a Poly(dT) sequence that are sequentially connected; wherein,
the sequencing primer binding sequence is configured to be complementarily paired with a primer upon sequencing;
the spatial position tag sequence is configured to mark the spatial position of the mRNA sample captured, and the spatial position tag sequences of the coding probes connected to the hydrophilic structures located within a same hydrophilic region are same, and the spatial position tag sequences of the coding probes connected to the hydrophilic structures located in different hydrophilic regions are different; and the spatial position tag sequence comprises a spatial position sub-tag sequence;
the UMI sequence is configured to specifically mark different mRNA samples; and
the Poly(dT) sequence is configured to capture the mRNA sample.

12. The spatial omics chip of claim 11, wherein the sequencing primer binding sequence comprises a linking group through which the sequencing primer binding sequence is connected to the hydrophilic structure, and the linking group comprises at least one of a carboxyl group, a thiol group and an amino group;
or, the sequencing primer binding sequence and the hydrophilic structure are connected together by at least one of an amino-carboxyl reaction, a thiol-thiol reaction, and an anhydride-amino reaction;
or, the sequencing primer binding sequence and the hydrophilic structure are connected together by at least one of an amide group and a disulfide bond.

13. The spatial omics chip according to claim 1, comprising a plurality of sub-chips distributed at intervals, wherein the plurality of sub-chips are distributed on the first substrate in a periodic array, and the sub-chip comprises a plurality of hydrophilic regions.

14. A rotary step-by-step sample injection chip, configured to perform step-by-step sample injection into the hydrophilic regions of the spatial omics chip according to claim 1, comprising: a second substrate, and a plurality of flow channels arranged on the same side of the second substrate and distributed at intervals, wherein the flow channels have a plurality of through holes penetrating the second substrate and distributed in a periodic array, each of the hydrophilic regions of the spatial omics chip corresponds to one of the through holes, a distance between the centers of two adjacent through holes is same as a distance between the centers of two adjacent hydrophilic regions corresponding to the two through holes correspond, and the through holes are configured to perform step-by-step sample injection into each of the hydrophilic structures of the hydrophilic regions corresponding to the through holes.

15. The rotary step-by-step sample injection chip according to claim 14, wherein an area of the through hole is less than or equal to an area of the hydrophilic region to which the through hole corresponds

16. The rotary step-by-step sample injection chip according to claim 15, comprising an effective sample injection region, wherein the rotary step-by-step sample injection chip is rotated around a center thereof at least once at a set angle, causing that at least a portion of the through holes of the rotary step-by-step sample injection chip completely overlaps before and after at least one rotation, and the completely overlapping through holes forming the effective sample injection region; when the spatial omics chip and the rotary step-by-step sample injection chip are matched and used, an orthographic projection of the through holes in the effective sample injection region on the first substrate overlaps with an orthographic projection of the hydrophilic region of the spatial omics chip on the first substrate;
wherein the effective sample injection region is a regular polygon having n sides, and n is an integer ≥3;
a distance between centerlines of two adjacent flow channels satisfies:
$${L}_{3} = {L}_{2} \times sin θ ;$$
wherein L₃ is a distance between two adjacent flow channels;
L₂ is a distance between the centers of two adjacent through holes;
θ is a rotation angle of the rotary step-by-step sample injection chip.

17. (canceled)

18. A synthesis method for a spatial position tag sequence, comprising:
injecting a sub-spatial position tag sequence multiple times through a through hole of the rotary step-by-step sample injection chip according to claim 14, and between two adjacent injections, rotating the rotary step-by-step sample injection chip at a set angle around a center thereof, such that at least part of the through holes of the rotary step-by-step sample injection chip completely overlap before and after the rotation, injecting the sub-spatial position tag sequence into a hydrophilic region of the spatial omics chip through the through holes in the overlapping region to form the spatial position tag sequence;
wherein the sub-spatial position tag sequences injected into a plurality of the through holes on the same flow channel are same, and the sub-spatial position tag sequences injected into the through holes on different flow channels are different.

19. The synthesis method according to claim 18, wherein injecting the sub-spatial position tag sequence into a hydrophilic region of the spatial omics chip through the through holes in the overlapping region comprises:
bonding the rotary step-by-step sample injection chip to a spatial omics chip comprising a first substrate, hydrophobic regions arranged on the same side of the first substrate, and a plurality of hydrophilic regions distributed in a periodic array and spaced apart from each other, wherein each hydrophilic region is surrounded by the hydrophobic regions, and the hydrophilic region comprises a hydrophilic structure configured to be connected to a coding probe, such that the hydrophilic region of the spatial omics chip corresponds to the through hole of the flow channel of the rotary step-by-step sample injection chip, wherein the hydrophilic structure of the hydrophilic region is connected to a nucleic acid sequence;
injecting different sub-spatial position tag sequences into different flow channels of the rotary step-by-step sample injection chip respectively, such that the sub-spatial position tag sequences enter into the hydrophilic region of the spatial omics chip through the through holes on the flow channel, and are connected to nucleic acid sequences on the hydrophilic structures.

20. A manufacturing method of a spatial omics chip according to claim 1, comprising:
covering the first substrate with a first photomask having a first light-transmitting region and a first light-shielding region, wherein the first light-transmitting region completely coincides with an orthographic projection of a region on the first substrate where the hydrophilic structure is to be formed on the first substrate;
under illumination conditions, using a hydrophilic monomer as a raw material, performing a graft polymerization reaction on a region on the first substrate that completely coincides with an orthographic projection of the first light-transmitting region to form a hydrophilic polymer, wherein the hydrophilic polymer comprises an active group capable of being connected to the coding probe, and forms the hydrophilic structure;
covering the first substrate with a second photomask having a second light-transmitting region and a second light-shielding region, wherein the second light-transmitting region completely coincides with an orthographic projection of a region on the first substrate where the hydrophobic structure is to be formed on the first substrate;
under illumination conditions, using a hydrophobic monomer as a raw material,
performing a graft polymerization reaction on a region on the first substrate that completely coincides with the orthographic projection of the second light-transmitting region to form a hydrophobic polymer, wherein the hydrophobic polymer comprises the hydrophobic group and forms a hydrophobic structure, and the hydrophobic structure forms the hydrophobic region.

21. The manufacturing method according to claim 20, further comprising sequentially ligating a sequencing primer binding sequence, a spatial position tag sequence, a UMI sequence and a Poly(dT) sequence at an end of the hydrophilic structure away from the first substrate;
wherein the spatial position tag sequence is formed using a synthesis method of the spatial position tag sequence comprising:
injecting a sub-spatial position tag sequence multiple times through a through hole of a rotary step-by-step sample injection chip, and between two adjacent injections, rotating the rotary step-by-step sample injection chip at a set angle around a center thereof, such that at least part of the through holes of the rotary step-by-step sample injection chip completely overlap before and after the rotation, injecting the sub-spatial position tag sequence into a hydrophilic region of the spatial omics chip through the through holes in the overlapping region to form the spatial position tag sequence, wherein the rotary step-by-step sample injection chip is configured to perform step-by-step sample injection into the hydrophilic regions of the spatial omics chip, and comprises: a second substrate, and a plurality of flow channels arranged on the same side of the second substrate and distributed at intervals, wherein the flow channels have a plurality of through holes penetrating the second substrate and distributed in a periodic array, each of the hydrophilic regions of the spatial omics chip corresponds to one of the through holes, a distance between the centers of two adjacent through holes is same as a distance between the centers of two adjacent hydrophilic regions corresponding to the two through holes correspond, and the through holes are configured to perform step-by-step sample injection into each of the hydrophilic structures of the hydrophilic regions corresponding to the through holes;
wherein the sub-spatial position tag sequences injected into a plurality of the through holes on the same flow channel are same, and the sub-spatial position tag sequences injected into the through holes on different flow channels are different.
